(19) United States
(12) Patent Application Publication
(10) Pub. No.: US 2024/0094176 A1
(43) Pub. Date: Mar. 21, 2024

(54) GAS ANALYSIS DEVICE, FLUID CONTROL SYSTEM, GAS ANALYSIS PROGRAM, AND GAS ANALYSIS METHOD

(71) Applicant: HORIBA STEC, Co., Ltd., Kyoto (JP)

(72) Inventors: Motonobu TAKAHASHI, Kyoto (JP); Masakazu MINAMI, Kyoto (JP); Yuhei SAKAGUCHI, Kyoto (JP)

(73) Assignee: HORIBA STEC, Co., Ltd., Kyoto (JP)

(21) Appl. No.: 18/369,332

(22) Filed: Sep. 18, 2023

(30) Foreign Application Priority Data
Sep. 21, 2022 (JP) 2022-150243

(51) Int. Cl. G01N 33/00; B01L 3/00; B01L 5/00

(52) U.S. Cl. CPC G01N 33/0013; B01L 3/502761; B01L 5/00; B01L 2200/16; B01L 2400/082

(57) ABSTRACT

The present invention brings an actual concentration of a process gas closer to an ideal concentration, and a gas analysis device that is used in a fluid control system that controls a process gas obtained by vaporizing a liquid material or a solid material, the gas analysis device including: a first concentration calculation unit that calculates a concentration of the process gas; a second concentration calculation unit that calculates a concentration of a by-product gas at least generated in a side reaction that is a reaction different from a main reaction for generating the process gas; a comparison unit that compares a first actual concentration that is the concentration of the process gas calculated by the first concentration calculation unit with a first ideal concentration, and compares a second actual concentration that is the concentration of the by-product gas calculated by the second concentration calculation unit with a second ideal concentration.

### CROSS-REFERENCE TO RELATED APPLICATIONS

The present application claims priority to Japan Patent Application No. 2022-150243 filed Sep. 21, 2022, which is incorporated herein by reference in its entirety.

### BACKGROUND OF THE INVENTION

### 1. Technical Field

The present invention relates to a gas analysis device, a fluid control system, a gas analysis program, and a gas analysis method.

### 2. Description of the Related Art

There are some conventional semiconductor manufacturing systems configured to generate a process gas by vaporizing a liquid material or a solid material, and to supply the process gas to a chamber.

In such a system, not only the main reaction for generating a process gas by vaporizing a liquid material or a solid material, but also side reactions, such as liquefaction or decomposition of the process gas, different from the main reaction take place.

For this reason, the actual concentration (hereinafter, referred to as an actual concentration) of the process gas is not the same as an ideal concentration (hereinafter, referred to as an ideal concentration) achieved when the main reaction takes place most favorably. In other words, the ratio of the ideal concentration with respect to the actual concentration (hereinafter, referred to as a vaporization efficiency) varies depending on what kind of side reaction takes place.

However, because it is difficult to identify the type of side reactions taking place, it has been a conventional technical common sense to increase the amount of the liquid material or the solid material used, rather than to improve the vaporization efficiency, in order to adjust the concentration of the process gas to an intended concentration.

When the material is expensive, however, the cost spent in the manufacturing process increases, and such an increase leads to an increase in the cost of the final product, disadvantageously.

### PRIOR ART DOCUMENT

### Patent Document

JP 2000-217894 A

### SUMMARY OF THE INVENTION

Therefore, the present invention has been made to solve the problem described above, and a main object of the present invention is to improve the vaporization efficiency so as to bring the actual concentration of a process gas closer to the ideal concentration.

In other words, a gas analysis device according to the present invention is a gas analysis device that is used in a fluid control system that controls a process gas obtained by vaporizing a liquid material or a solid material, the gas analysis device including: a first concentration calculation unit that calculates a concentration of the process gas; a second concentration calculation unit that calculates a concentration of a by-product gas at least generated in a side reaction that is a reaction different from a main reaction for generating the process gas; a comparison unit that compares a first actual concentration that is the concentration of the process gas calculated by the first concentration calculation unit with a first ideal concentration that is the concentration of the process gas achieved when the main reaction takes place most favorably, and compares a second actual concentration that is the concentration of the by-product gas calculated by the second concentration calculation unit with a second ideal concentration that is the concentration of the by-product gas achieved when the main reaction takes place most favorably; and an output unit that determines a parameter to be changed, that is a parameter a setting value of which is to be changed, among parameters set in devices included in the fluid control system, based on comparison results of the comparison unit, and outputs the parameter to be changed.

With the gas analysis device having the configuration described above, because there is a correlation between a quantitative relationship between the first actual concentration and the first ideal concentration and a quantitative relationship between the second actual concentration and the second ideal concentration, and the side reaction taking place, the parameter to be changed is determined and output based on the comparison results of these concentrations. Therefore, by changing the setting value of the parameter to be changed, the vaporization efficiency can be improved, which, in turn, enables the actual concentration of the process gas to be brought closer to the ideal concentration.

Preferably, the parameter to be changed is a heating temperature of a pipe through which the process gas flows, a heating temperature of a vaporizer that vaporizes the liquid material or the solid material, a flow rate setting of the vaporizer, or a concentration of the liquid material.

By changing the setting values of these parameters to be changed, when liquefaction or decomposition of the process gas or redissolution of the material occurs, it is possible to suppress the progress of such a side reaction, so that vaporization efficiency can be improved.

When the comparison result of the comparison unit indicates that the first actual concentration is lower than the first ideal concentration, and that the difference between the second actual concentration and the second ideal concentration is equal to or less than a threshold, it is highly likely that liquefaction of the process gas is taking place.

Therefore, in this case, preferably, the output unit outputs the heating temperature for the pipe, as the parameter to be changed.

In this case, by increasing the heating temperature for the pipe, it is possible to suppress the liquefaction of the process gas.

When the comparison result of the comparison unit indicates that the first actual concentration is lower than the first ideal concentration, the difference between the second actual concentration and the second ideal concentration is greater than the threshold, and the second actual concentration is higher than the second ideal concentration, it is highly likely that decomposition of the process gas is taking place.

Therefore, in this case, the output unit preferably outputs the heating temperature for the vaporizer or the flow rate setting of the vaporizer as the parameter to be changed.

In this case, by increasing the flow rate setting of the carrier gas, decreasing the heating temperature for the vaporizer, or decreasing the flow rate setting of the vaporizer, it is possible to suppress decomposition of the process gas.

When the comparison result of the comparison unit indicates that the first actual concentration is lower than the first ideal concentration, the difference between the second actual concentration and the second ideal concentration is greater than the threshold, and the second actual concentration is lower than the second ideal concentration, it is highly likely that a side reaction of either liquefaction or decomposition of the process gas, or redissolution of the material has taken place.

Therefore, in this case, preferably the output unit outputs the heating temperature for the pipe, the heating temperature for the vaporizer, the flow rate setting of the vaporizer, or the concentration of the liquid material, as the parameter to be changed.

In this manner, it is possible to suppress the progress of the side reaction taking place, by appropriately changing the setting value of the parameter to be changed.

Preferably, the gas analysis device further includes an adjustment unit that receives the parameter to be changed from the output unit, and that adjusts the setting value of the parameter to be changed.

With such a configuration, it is possible to automate an adjustment of the setting value of the parameter to be changed.

Preferably, the output unit outputs the parameter to be changed visually recognizably.

With such a configuration, it is possible to have the user recognize the parameter to be changed, in order to improve the vaporization efficiency. The user can then improve the vaporization efficiency by changing the setting value of the parameter to be changed, based on his/her experience, for example.

Preferably, the first concentration calculation unit and the second concentration calculation unit calculate the respective concentrations based on an output signal from the common photodetector.

In this manner, the concentrations of the process gas and of the by-product gas can be calculated using the common photodetector, so that reductions in the size as well as the manufacturing cost of the device can be achieved.

According to another aspect of the present invention, a fluid control system includes a vaporizer that vaporizes the liquid material or the solid material, a fluid controller that controls the process gas, the liquid material, or the carrier gas, and the gas analysis device.

Furthermore, a gas analysis program according to the present invention is used in a fluid control system that controls a process gas obtained by vaporizing a liquid material or a solid material, the gas analysis program causing a computer to exert functions as: a first concentration calculation unit that calculates a concentration of the process gas; a second concentration calculation unit that calculates a concentration of a by-product gas at least generated in a side reaction that is a reaction different from a main reaction for generating the process gas; a comparison unit that compares a first actual concentration that is the concentration of the process gas calculated by the first concentration calculation unit with a first ideal concentration that is the concentration of the process gas achieved when the main reaction takes place most favorably, and compares a second actual concentration that is the concentration of the by-product gas calculated by the second concentration calculation unit with a second ideal concentration that is the concentration of the by-product gas achieved when the main reaction takes place most favorably; and an output unit that determines a parameter to be changed, that is a parameter a setting value of which is to be changed, among parameters set in devices included in the fluid control system, based on comparison results of the comparison unit, and outputs the parameter to be changed.

In addition, a gas analysis method according to the present invention is a gas analysis method used in a fluid control system that controls a process gas obtained by vaporizing a liquid material or a solid material, the gas analysis method including: a first concentration calculation step of calculating a concentration of the process gas; a second concentration calculation step of calculating a concentration of a by-product gas at least generated in a side reaction that is a reaction different from a main reaction for generating the process gas; a comparison step of comparing a first actual concentration that is the concentration of the process gas calculated by the first concentration calculation unit with a first ideal concentration that is the concentration of the process gas achieved when the main reaction takes place most favorably, and comparing a second actual concentration that is the concentration of the by-product gas calculated by the second concentration calculation unit with a second ideal concentration that is the concentration of the by-product gas achieved when the main reaction takes place most favorably; and an output step of determining a parameter to be changed, that is a parameter a setting value of which is to be changed, among parameters set in devices included in the fluid control system, based on comparison results of the comparison step, and of outputting the parameter to be changed.

With such a gas analysis program and gas analysis method, it is possible to achieve the same operational effects as those achieved by the gas analysis device described above.

Further, a gas analysis device according to the present invention is a gas analysis device that analyzes a compound gas generated in a main reaction in which a solid material is vaporized, and a by-product gas generated in a side reaction that is different from the main reaction, the gas analysis device including: a first concentration calculation unit that calculates a concentration of the compound gas; a second concentration calculation unit that calculates a concentration of the by-product gas; a comparison unit that compares a first actual concentration that is the concentration of the compound gas calculated by the first concentration calculation unit, with a first ideal concentration that is the concentration of the compound gas achieved when the main reaction takes place most favorably, and compares a second actual concentration that is the concentration of the by-product gas calculated by the second concentration calculation unit with a second ideal concentration that is the concentration of the by-product gas achieved when the main reaction takes place most favorably; and an output unit that outputs an analysis result based on comparisons performed by the comparison unit.

With the gas analysis device having the configuration described above, because the side reaction currently taking place is correlated with the quantitative relationship between the first actual concentration and the first ideal concentration and the quantitative relationship between the second actual concentration and the second ideal concentration, these concentrations are compared and the analysis result is output, so that it is made easier to identify a cause when there is a difference between the first actual concentration and the first ideal concentration.

As a result, it becomes easier to determine the parameter having a setting value that requires a change to improve the vaporization efficiency. With this, it becomes possible to improve the vaporization efficiency, and to bring the actual concentration of the process gas closer to the ideal concentration.

A gas analysis program according to the present invention is a program used in a gas analysis device that analyzes a compound gas generated in a main reaction that is vaporization of a solid material and a by-product gas generated in a side reaction that is different from the main reaction, the gas analysis program causing a computer to execute functions as: a first concentration calculation unit that calculates a concentration of the compound gas; a second concentration calculation unit that calculates a concentration of the by-product gas; a comparison unit that compares a first actual concentration that is the concentration of the compound gas calculated by the first concentration calculation unit, with a first ideal concentration that is the concentration of the compound gas achieved when the main reaction takes place most favorably, and compares a second actual concentration that is the concentration of the by-product gas calculated by the second concentration calculation unit with a second ideal concentration that is the concentration of the by-product gas achieved when the main reaction takes place most favorably; and an output unit that outputs an analysis result based on comparisons performed by the comparison unit.

Furthermore, a gas analysis method according to the present invention is a gas analysis method of analyzing a compound gas generated in a main reaction in which a solid material is vaporized, and a by-product gas generated in a side reaction that is different from the main reaction, the gas analysis method including: an analysis step of comparing a first actual concentration that is a calculated concentration of the compound gas with a first ideal concentration that is the concentration of the compound gas achieved when the main reaction takes place most favorably, and compares a second actual concentration that is a calculated concentration of the by-product gas with a second ideal concentration that is the concentration of the by-product gas achieved when the main reaction takes place most favorably; and an output step of outputting an analysis result based on comparisons performed at the analysis step.

Furthermore, a gas analysis device according to the present invention is a gas analysis device that analyzes a compound gas and a by-product gas generated in a main reaction that is vaporization of a solid material, the gas analysis device including: a first concentration calculation unit that calculates a concentration of the compound gas; a second concentration calculation unit that calculates a concentration of the by-product gas; and an output unit that outputs a first actual concentration that is a concentration of the compound gas calculated by the first concentration calculation unit, and a first ideal concentration that is a concentration of the compound gas when the main reaction takes place most favorably, in a comparable manner, and outputs a second actual concentration that is a concentration of the by-product gas calculated by the second concentration calculation unit, and a second ideal concentration that is the concentration of the by-product gas achieved when the main reaction takes place most favorably, in a comparable manner.

According to the present invention described above, the vaporization efficiency can be improved, and the actual concentration of the process gas can be brought closer to the ideal concentration.

### BRIEF DESCRIPTION OF THE DRAWINGS

FIG. 1 is a schematic diagram illustrating a fluid control system in which a gas analysis device according to a first embodiment of the present invention is incorporated;

FIG. 2 is a schematic indicating chemical reaction formulas for explaining types of side reactions of the embodiment;

FIG. 3 is a schematic diagram illustrating a configuration of a concentration monitor according to the embodiment;

FIG. 4 is a functional block diagram illustrating functions of an information processing unit according to the embodiment;

FIG. 5 is a flowchart illustrating a former half of an operation performed by an information processing unit according to the embodiment;

FIG. 6 is a flowchart illustrating a latter half of the operation of the information processing unit according to the embodiment;

FIG. 7 is a functional block diagram illustrating functions of an information processing unit according to a modification of the first embodiment;

FIG. 8 is a schematic diagram illustrating a fluid control system in which a gas analysis device according to a modification of the first embodiment is incorporated;

FIG. 9 is a flowchart illustrating a latter half of an operation of an information processing unit according to a modification of the first embodiment;

FIG. 10 is a schematic diagram illustrating a fluid control system in which a gas analysis device according to a modification of the first embodiment is incorporated;

FIG. 11 is a flowchart illustrating a latter half of the operation of the information processing unit according to a modification of the first embodiment;

FIG. 12 is a schematic diagram for explaining an aspect in which a solid material is used in a modification of the first embodiment;

FIG. 13 is a schematic indicating chemical reaction formulas for explaining the type of side reaction in an example in which the solid material is used;

FIG. 14 is a flowchart for explaining the operation of the information processing unit in the example in which the solid material is used;

FIG. 15 is a functional block diagram illustrating functions of an information processing unit according to a second embodiment of the present invention;

FIG. 16 is a flowchart for explaining an operation of the information processing unit according to the embodiment; and

FIG. 17 is a functional block diagram illustrating functions of an information processing unit according to a modification of the second embodiment.

### DETAILED DESCRIPTION

### First Embodiment

A gas analysis device according to a first embodiment of the present invention will now be explained with reference to some drawings.

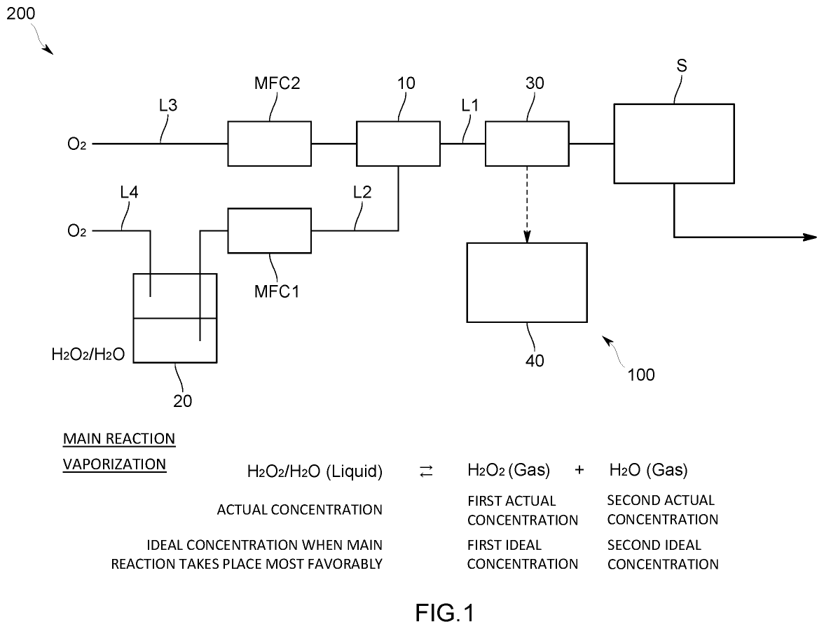

As illustrated in FIG. 1, a gas analysis device 100 according to the present embodiment is a building component of a fluid control system 200 that controls a gas to be supplied to a predetermined gas supply space S, and is configured to measure the concentration of the gas.

To begin with, the fluid control system 200 will be explained. As illustrated in FIG. 1, the fluid control system supplies a process gas to a process chamber that is the gas supply space S of a semiconductor manufacturing apparatus, for example. Specifically, the fluid control system 200 includes a vaporizer 10 that vaporizes a liquid material or a solid material, and a gas supply line L1 for supplying the process gas resultant of vaporizing the liquid material or the solid material in the vaporizer 10, to the process chamber S.

The vaporizer 10 is configured to vaporize a liquid material or a solid material by heating and/or reducing the pressure, and may also be configured to vaporize a liquid material that is a mixture of a chemical compound and water, to vaporize a liquid material obtained by dissolving a solid material into a liquid, or to sublimate a solid material.

In the example explained below, it is assumed that an aqueous solution prepared by mixing hydrogen peroxide (H₂O₂) with water (H₂O) having the concentration of hydrogen peroxide adjusted to a desired concentration is used as a liquid material.

The vaporizer 10 according to the present embodiment includes a heater (not illustrated) that heats the liquid material, and a nozzle (not illustrated) that ejects to vaporize the liquid material. A specific configuration of the vaporizer 10 is, however, not limited thereto. For example, the vaporizer 10 may be a bubbler that vaporizes a liquid material while supplying a carrier gas into a tank containing the liquid material, or may be of a baking vaporizer that vaporizes a liquid material or a solid material by placing a tank containing the liquid material or the solid material in a thermostatic chamber.

A material feed line L2 for guiding the liquid material stored in the reservoir 20 and a carrier gas feed line L3 for guiding the carrier gas are connected to the vaporizer 10, and a pressure-feeding gas feed line L4 for guiding pressure-fed gas is guided is connected to the reservoir 20. The material feed line L2 is provided with a first mass flow controller MFC1, as a fluid controller that controls the flow rate of the liquid material, and the carrier gas feed line L3 is provided with a second mass flow controller MFC2 as a fluid controller that controls the flow rate of the carrier gas. In the example explained herein, oxygen is used as the carrier gas and the pressure-feeding gas, but nitrogen, argon, hydrogen, or the like may also be used, depending on the type of the liquid material.

As illustrated in FIG. 1, the gas supply line L1 connects the vaporizer 10 to the gas supply space S, and through which a process gas and a by-product gas generated by a main reaction in the vaporizer 10 flow. In this embodiment, the process gas is hydrogen peroxide gas, and the by-product gas is H₂O gas. In addition to these gases, oxygen that is the carrier gas and the pressure-feeding gas described above also flow through the gas supply line L1.

The by-product gas in this embodiment is generated as a by-product of the main reaction as described above, but may also be generated by a side reaction that is different from the main reaction. In other words, the concentration of the by-product gas can vary depending on what kind of side reaction takes place, and may also cause the concentration of the process gas to vary.

Therefore, the present invention resides in the finding of a technical significance in the monitoring of a by-product gas concentration, which will be described in detail below.

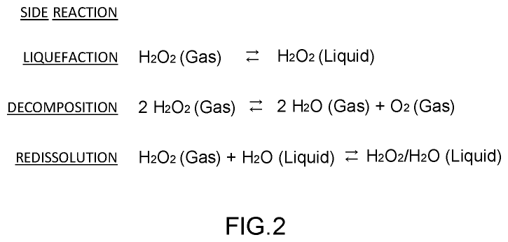

As illustrated in FIG. 2, examples of the side reaction according to the present embodiment include liquefaction of the process gas (hydrogen peroxide gas), decomposition of the process gas (hydrogen peroxide gas), and redissolution of the process gas (hydrogen peroxide gas) in water resultant of the liquefaction of the by-product gas (H₂O gas).

The gas analysis device 100 incorporated in the fluid control system described above will now be explained.

As illustrated in FIG. 1, the gas analysis device 100 includes a concentration monitor 30 provided in the gas supply line L1 and an information processing unit 40 that acquires an output signal from the concentration monitor 30. The concentration monitor 30 does not need to be provided in the gas supply line L1, and may be provided, for example, in a branch line branched out from the gas supply line L1.

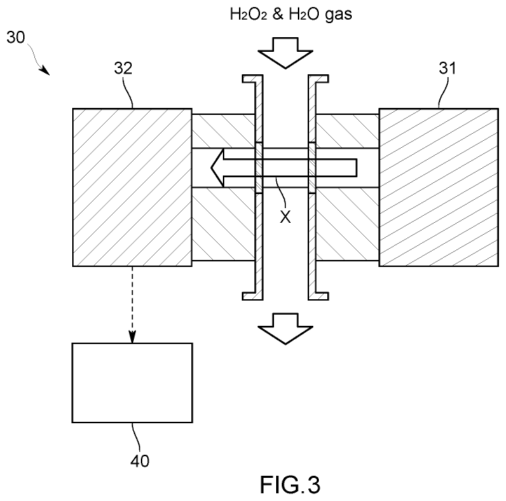

The concentration monitor 30 analyzes a component to be measured in a gas, using an infrared absorption method. Specifically, as illustrated in FIG. 3, the concentration monitor 30 includes a light source unit 31 in which a light source for irradiating the gas with infrared light X is housed, and a detector unit 32 in which a photodetector for detecting the infrared light X passed through the gas is housed, and is configured to output a light intensity signal corresponding to the infrared light X detected by the photodetector to the information processing unit 40 as an output signal.

The information processing unit 40 is a general-purpose or dedicated computer including a CPU, a memory, an AD converter, a DA converter, and the like, and may be provided integrally with the concentration monitor 30 or separately from the concentration monitor 30.

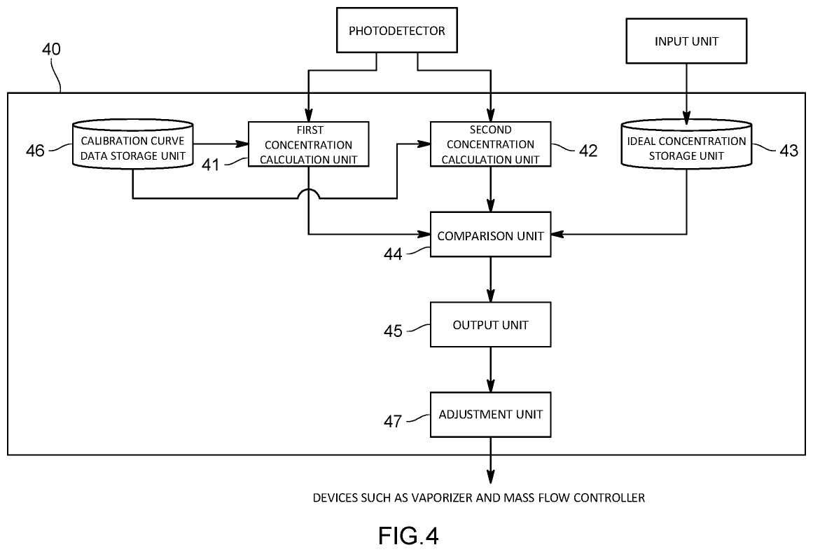

The information processing unit 40 exerts functions as a first concentration calculation unit 41, a second concentration calculation unit 42, an ideal concentration storage unit 43, a comparison unit 44, and an output unit 45 as illustrated in FIG. 4, by causing the CPU and its peripheral devices to cooperate with one another, in accordance with a gas analysis program stored in a predetermined area of the memory.

The concentration of the gas described below may be a concentration of the component in the gas or a partial pressure of the gas.

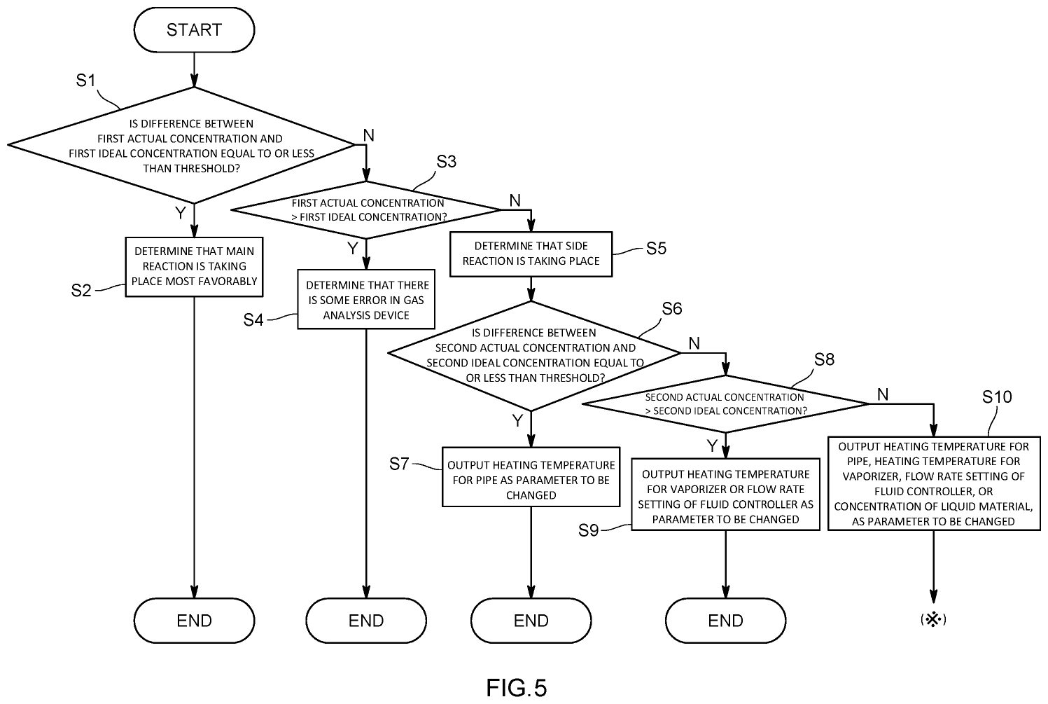

An operation of the information processing unit 40 according to the present embodiment will now be explained, with reference to FIGS. 4 and 5, also as an explanation of the function of each unit.

The first concentration calculation unit 41 calculates the concentration of the hydrogen peroxide gas that is the process gas (hereinafter, also referred to as a first actual concentration). Specifically, the first concentration calculation unit 41 receives a light intensity signal that is an output signal from the photodetector, and performs an operation on the value indicated by the light intensity signal, to calculate the concentration of the hydrogen peroxide gas contained in the gas flowing through the gas supply line L1, as the first actual concentration. Note that this operation uses first calibration curve data indicating the relationship between the value indicated by the light intensity signal and the first actual concentration, and that the first calibration curve data is stored in a calibration curve data storage unit 46 set in a predetermined area of the memory (see FIG. 4).

The second concentration calculation unit 42 calculates the concentration of the H₂O gas that is a by-product gas (hereinafter, also referred to as a second actual concentration). Specifically, the second concentration calculation unit 42 receives a light intensity signal that is an output signal from the photodetector, and performs an operation on the value indicated by the light intensity signal, to calculate the concentration of the H₂O gas contained in the gas flowing through the gas supply line L1, as the second actual concentration. Note that this operation uses second calibration curve data indicating the relationship between the value indicated by the light intensity signal and the second actual concentration, and that the second calibration curve data is stored in the calibration curve data storage unit 46 set in a predetermined area of the memory (see FIG. 4).

In the present embodiment, the first concentration calculation unit 41 and the second concentration calculation unit 42 are configured to calculate the first actual concentration and the second actual concentration, respectively, based on output signals from the common photodetector. In this manner, reductions in the size as well as the manufacturing cost of the device can be achieved. However, it is also possible to configure the first concentration calculation unit 41 and the second concentration calculation unit 42 to calculate the first actual concentration and the second actual concentration, respectively, based on output signals from the different photodetectors, respectively.

The ideal concentration storage unit 43 is set in a predetermined area of the memory, and stores therein a first ideal concentration that is the ideal concentration of the hydrogen peroxide gas achieved when the main reaction described above takes place most favorably, and a second ideal concentration that is the concentration of the H₂O gas also achieved when the main reaction described above takes place most favorably.

The first ideal concentration may be obtained in advance, through a calculation, e.g., before control process of the fluid control system 200 starts. Specifically, the first ideal concentration can be calculated based on a concentration of hydrogen peroxide (specifically, a volume fraction of hydrogen peroxide) theoretically obtained from a titration concentration obtained by actually measuring, e.g., titrating, the concentration of hydrogen peroxide contained in the aqueous solution stored in the reservoir 20, and a total flow rate (combined flow rate of the flow rate of hydrogen peroxide gas, the flow rate of H₂O gas, and the flow rate of oxygen gas) of gas flowing through the concentration monitor 30.

An example of a specific method for calculating the first ideal concentration is as follows.

To begin with, the molar concentration of the hydrogen peroxide water in the aqueous solution stored in the reservoir 20 is measured using titration that uses the oxidation-reduction reaction, and the mass percentage concentration of the hydrogen peroxide water is determined by measuring the weight of the aqueous solution.

The weight of the hydrogen peroxide water fed into the vaporizer 10 per unit time, and the weight of the water fed into the vaporizer 10 per unit time are then obtained, based on the mass percent concentration and the flow rate setting of the first mass flow controller MFC1 described above.

By converting these weights into volumes, the volume of the hydrogen peroxide gas generated in the vaporizer 10 and flowing into the concentration monitor 30 per unit time (that is, the volumetric flow rate of the hydrogen peroxide gas), and the volume of the H₂O gas generated in the vaporizer 10 and flowing into the concentration monitor 30 per unit time (that is, the volumetric flow rate of the H₂O gas) are obtained.

The volume of the carrier gas (oxygen) fed into the vaporizer 10 and flowing into the concentration monitor 30 per unit time (that is, the volumetric flow rate of the carrier gas) is obtained from the flow rate setting of the second mass flow controller MFC2.

In this manner, the volumetric flow rate of the hydrogen peroxide gas, the volumetric flow rate of the H₂O gas, and the volumetric flow rate of the oxygen gas flowing through the concentration monitor 30 are obtained, so that the flow rate of the hydrogen peroxide gas with respect to the total flow rate thereof can be used as the first ideal concentration.

A calculation of the first ideal concentration is not limited thereto, and various calculation methods may be used, based on technical common knowledge of those skilled in the art.

This theoretical concentration is the concentration of the hydrogen peroxide gas assuming that 100% of the aqueous solution stored in the reservoir 20 is vaporized, that is, the concentration of the hydrogen peroxide gas when only the main reaction described above takes place.

Although this theoretical concentration may be used as the first ideal concentration, in the present embodiment, the first ideal concentration is set considering the fact that compound gas decreases somewhat considerably due to factors such as condensation in the process of travelling from the reservoir 20 to the concentration monitor 30. In other words, because there is a difference between the theoretical concentration and the concentration measured by the concentration monitor 30 (hereinafter, referred to as an effective concentration), a ratio of the effective concentration with respect to the theoretical concentration is obtained in advance, and this ratio is multiplied to the theoretical concentration, to establish the resultant concentration as the first ideal concentration. It is, however, possible to use the effective concentration as the first ideal concentration, without obtaining this ratio.

In the same manner as the first ideal concentration, the second ideal concentration may be obtained in advance, through a calculation, e.g., before the control process of the fluid control system 200 starts. Specifically, the concentration can be calculated based on a theoretical concentration of H₂O (specifically, the volume fraction of H₂O) calculated from the titration concentration described above, and from the total flow rate of the gas flowing through the concentration monitor 30. The concentration resultant of multiplying the ratio described above to the theoretical concentration is used the second ideal concentration. It is also possible to use the concentration of the H₂O gas measured in advance by the concentration monitor 30, before the control process of the fluid control system 200 starts, as the second ideal concentration. As an example of a specific method for calculating the second ideal concentration, the same method as that used for calculating the first ideal concentration may be used.

The first ideal concentration and the second ideal concentration thus calculated are input from the external via an input unit, for example, and stored in the ideal concentration storage unit 43. It is also possible to provide the information processing unit 40 with the function as an ideal concentration calculation unit that calculates the first ideal concentration and the second ideal concentration, and to store the first ideal concentration and the second ideal concentration calculated by the ideal concentration calculation unit in the ideal concentration storage unit 43.

The comparison unit 44 compares the first actual concentration and the first ideal concentration, and compares the second actual concentration and the second ideal concentration. Specifically, the comparison unit 44 determines a quantitative relationship between the first actual concentration and the first ideal concentration, and determines a quantitative relationship between the second actual concentration and the second ideal concentration.

More specifically, as illustrated in FIG. 5, to begin with, the comparison unit 44 compares the first actual concentration and the first ideal concentration, and determines whether the difference between the first actual concentration and the first ideal concentration is equal to or less than a threshold (S1).

If the difference between the first actual concentration and the first ideal concentration is equal to or less than the predetermined threshold, the comparison unit 44 determines that the main reaction is taking place most favorably (S2).

By contrast, if the difference between the first actual concentration and the first ideal concentration is greater than the predetermined threshold in S1, the comparison unit 44 determines the quantitative relationship between the first actual concentration and the first ideal concentration (S3), and determines whether a side reaction that is different from the main reaction is taking place, or whether there is any error in the gas analysis device 100 (S4, S5).

Specifically, if the first actual concentration is higher than the first ideal concentration in S3, the comparison unit 44 determines that there is some error in the device (S4). Examples of the errors include a calibration error and various errors in the settings, e.g., the first calibration curve data and the second calibration curve data, described above.

By contrast, if the first actual concentration is lower than the first ideal concentration in S3, the comparison unit 44 determines that a side reaction that is different from the main reaction is taking place (S5).

Note that the determinations in S2, S4, and S5 described above are not necessarily need to be performed by the comparison unit 44, and may be performed by a user, for example, or these determination steps may also be even omitted.

If it is determined that the first actual concentration is lower than the first ideal concentration in S3 described above, it can be said that the ratio of the first actual concentration with respect to the first ideal concentration (hereinafter, also referred to as vaporization efficiency) is lower than 100%.

To address this point, the fluid control system 200 according to the present embodiment is enabled to change the setting values of the parameters in various devices included in the fluid control system 200, depending on the side reactions that are presumably taking place, in an attempt to bring the vaporization efficiency closer to 100%.

More specifically, after determining that the first actual concentration is lower than the first ideal concentration in S3 described above, the comparison unit 44 compares the second actual concentration with the second ideal concentration, and determines whether the difference between the second actual concentration and the second ideal concentration is equal to or less than a threshold (S6).

Based on the comparison result of the comparison unit 44, the output unit 45 determines the parameter to be changed, which is a parameter the setting value of which should be changed to suppress the side reaction, among the parameters set in the devices included in the fluid control system 200, and outputs the parameter. Note that the parameter output from the output unit 45 is a signal for distinguishing the determined parameter to be changed, from the other parameters to be changed, e.g., a signal indicating the name or the like of the parameter to be changed.

Note that the devices included in the fluid control system 200 include a device for vaporizing a liquid material or a solid material, and a device for guiding the generated process gas to the process chamber S. Examples of the devices include the reservoir 20 for storing a liquid material, the first mass flow controller MFC1 for controlling the flow rate of the liquid material, the second mass flow controller MFC2 for controlling the flow rate of the carrier gas, the vaporizer 10, the pipes (not illustrated) forming the gas supply line L1, and the heater (not illustrated) for heating the pipe.

In the present embodiment, a storage unit such as an internal memory or an external memory of the information processing unit 40 stores therein determination information such as a table including candidates of the results of the comparisons in S6 and S8 by the comparison unit 44, and one or a plurality of parameters to be changed that are associated with the candidates, and the output unit 45 determines the parameter to be changed, using the determination information and the comparison result in each of the steps described above performed by the comparison unit 44, and outputs the parameter to be changed. Note that the storage unit described above may be a component of the information processing unit 40, may be a component of a computer that is different from the information processing unit 40 included in the fluid control system 200, or may be provided outside the fluid control system 200, such as a client server.

If the result of the comparison performed by the comparison unit 44 in S6 indicates that the difference between the second actual concentration and the second ideal concentration is equal to or less than the threshold, it is highly likely that the process gas flowing through the gas supply line L1 has liquefied, as a side reaction.

Therefore, when this is applicable, that is, if the comparison result of the comparison unit 44 in S3 indicates that the first actual concentration is lower than the first ideal concentration, and the result of the comparison of the comparison unit 44 in S6 indicates that the difference between the second actual concentration and the second ideal concentration is equal to or less than the threshold, the output unit 45 outputs the heating temperature for the pipe forming the gas supply line L1, as a parameter to be changed (S7).

Accordingly, by increasing the heating temperature for the pipe, it is possible to suppress liquefaction, which is presumed to be taking place as a side reaction.

In such a configuration, as illustrated in FIG. 4, the information processing unit 40 according to the present embodiment further includes a function of an adjustment unit 47 that receives the parameter to be changed from the output unit 45, and that adjusts the setting value of the parameter to be changed.

In other words, when the output unit 45 outputs the heating temperature for the pipe as the parameter to be changed, the adjustment unit 47 adjusts the heating temperature to a target temperature calculated based on the vaporization efficiency, for example, or increases the heating temperature by a predetermined constant temperature.

If the process returns to S7 after the heating temperature is adjusted (raised), in other words, if it is determined that the difference between the second actual concentration and the second ideal concentration is equal to or less than the threshold in S6, the output unit 45 may be configured to output the heating temperature for the pipe as the parameter to be changed again.

By contrast, as illustrated in FIG. 5, if the comparison result in S6 indicates that the difference between the second actual concentration and the second ideal concentration is greater than the threshold, the comparison unit 44 determines a quantitative relationship between the second actual concentration and the second ideal concentration (S8).

If the comparison result of the comparison unit 44 in S8 indicates that the second actual concentration is higher than the second ideal concentration, is it is highly likely that decomposition of the process gas flowing through the gas supply line L1 is taking place, as a side reaction.

Therefore, when this is applicable, that is, if the comparison result of the comparison unit 44 in S3 indicates that the first actual concentration is lower than the first ideal concentration, the comparison result of the comparison unit 44 in S6 indicates that the difference between the second concentration and the second ideal concentration is greater than the threshold, and the comparison result of the comparison unit 44 in S8 indicates that the second actual concentration is higher than the second ideal concentration, the output unit 45 outputs at least one of the heating temperature for the vaporizer 10, the flow rate setting of the vaporizer 10, or the flow rate setting of the second mass flow controller MFC2, as a parameter to be changed (S9). In the configuration illustrated in FIG. 1, the flow rate setting of the vaporizer 10 is the flow rate setting of the first mass flow controller MFC1.

The adjustment unit 47 is also configured to lower the heating temperature for the vaporizer 10, to reduce the flow rate setting of the first mass flow controller MFC1 that is the flow rate setting of the vaporizer 10, or increase the flow rate setting of the second mass flow controller MFC2, so as to be able to suppress decomposition of the process gas, which is presumed to be taking place as a side reaction.

As described above, in a case where there are a plurality of options as the parameters to be changed to suppress the side reaction, the output unit 45 may output the plurality of parameters to be changed at once, or may output the plurality of parameters to be changed one by one.

When there are a plurality of options for outputting the parameters to be changed, the output unit 45 according to the present embodiment is configured to sequentially output the parameters to be changed one by one, based on a preset priority.

More specifically, taking the operation in S9 as an example, the output unit 45 first outputs the flow rate setting of the second mass flow controller MFC2 as the parameter to be changed.

Once the flow rate setting of the second mass flow controller MFC2 is adjusted (increased) and then the process returns to S9 again, in other words, if it is determined that the second actual concentration is higher than the second ideal concentration again in S8, the output unit 45 then outputs the heating temperature for the vaporizer 10 as the parameter to be changed.

If the process returns to S9 again after the heating temperature for the vaporizer 10 is adjusted (decreased), that is, if it is determined that the second actual concentration is higher than the second ideal concentration again in S8, the output unit 45 finally outputs the flow rate setting of the first mass flow controller MFC1, which is the flow rate setting of the vaporizer 10, as the parameter to be changed.

If the flow rate setting of the first mass flow controller MFC1 is adjusted (increased) and then the process returns to S9 again, that is, if it is determined that the second actual concentration is higher than the second ideal concentration again in S8, the output unit 45 may be configured to output, for example, an error signal indicating that the second actual concentration is higher.

At this time, returning to the comparison in S8 in FIG. 5, if the comparison result of the comparison unit 44 in S8 indicates that the second actual concentration is lower than the second ideal concentration, it is highly likely that one or more of the side reactions of liquefaction of the process gas, decomposition of the process gas, and redissolution of the material is taking place.

Therefore, when this is applicable, that is, if the comparison result of the comparison unit 44 in S3 indicates that the first actual concentration is lower than the first ideal concentration, the comparison result of the comparison unit 44 in S6 indicates that the difference between the second concentration and the second ideal concentration is greater than the threshold, and the comparison result of the comparison unit 44 in S8 indicates that the second actual concentration is lower than the second ideal concentration, the output unit 45 outputs at least one of the heating temperature for the pipe forming the gas supply line L1, the heating temperature for the vaporizer 10, the flow rate setting of the first mass flow controller MFC1, which is the flow rate setting of the vaporizer 10, or the flow rate setting of the second mass flow controller MFC2, as a parameter to be changed (S10).

At this time, if one or more of the side reactions of liquefaction, decomposition, and redissolution are taking place, while the countermeasure for lowering the temperature of the pipe or the like is effective for decomposition, the countermeasure for raising the temperature of the pipe or the like is effective for liquefaction and redissolution; so that these respective countermeasures antagonize each other.

Therefore, in consideration of handling peroxide as a process gas, the adjustment unit 47 according to the present embodiment is configured to lower the temperature of the pipe or the like, to begin with, and, if there is no improvement in the side reaction, then to increase the temperature of the pipe or the like.

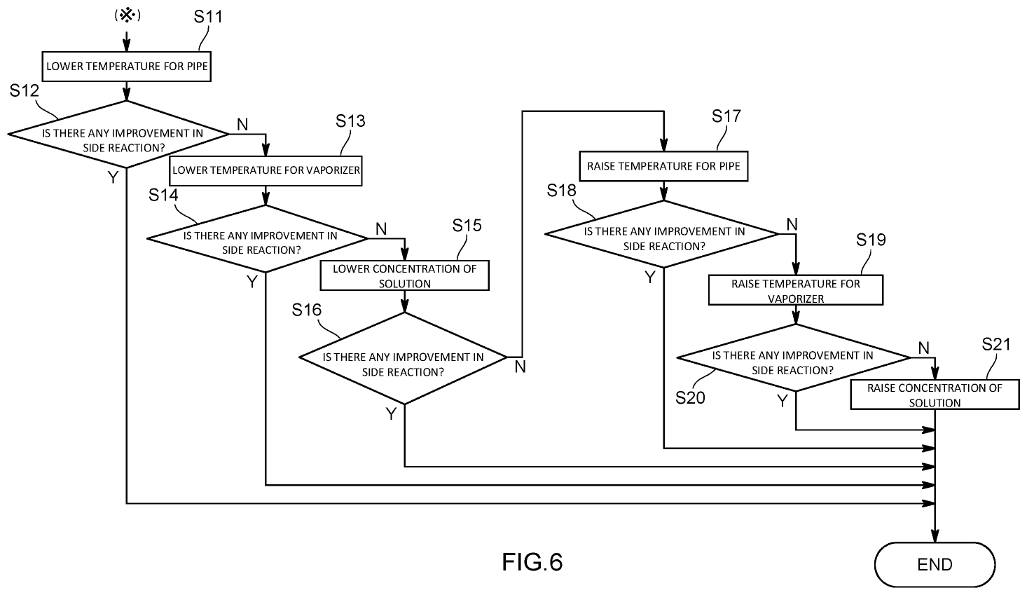

More specifically, as illustrated in FIG. 6, to begin with, the adjustment unit 47 lowers the heating temperature for the pipe forming the gas supply line L1 (S11).

The adjustment unit 47 then determines whether there is any improvement in the side reaction (S12), and ends the adjustment if there is some improvement in the side reaction.

As an example of the determination as to whether there is any improvement in the side reaction, for example, the adjustment unit 47 may determine that there is some improvement in the side reaction if it is determined that, after the adjustment unit 47 makes an adjustment such as that of a temperature, the second actual concentration becomes higher than the second ideal concentration in S8 in FIG. 5. The adjustment unit 47 may determine that there is no improvement in the side reaction if it is still determined that the second actual concentration is lower than the second ideal concentration.

As another example, after the adjustment unit 47 makes an adjustment such as that of a temperature, the sequence of the flowchart in FIG. 5 may be repeated; the adjustment unit 47 may then determine that there is no improvement in the side reaction if the process returns to S10 again; otherwise, the adjustment unit 47 determines that there is some improvement in the side reaction.

By contrast, if it is determined that there is no improvement in the side reaction in S12, the adjustment unit 47 lowers the heating temperature for the vaporizer 10 (S13).

The adjustment unit 47 then determines whether there is any improvement in the side reaction, in the same manner as in S12 (S14). If there is some improvement in the side reaction, the adjustment unit 47 ends the adjustment.

By contrast, if it is determined that there is no improvement in the side reaction in S14, the adjustment unit 47 controls to cause a concentration adjustment device (not illustrated) or the like to lower the concentration of the aqueous solution in the reservoir 20 (S15).

The adjustment unit 47 then determines whether there is any improvement in the side reaction as in S12 (S16), and ends the adjustment if there is some improvement in the side reaction.

By contrast, if it is determined that there is no improvement in the side reaction in S16, the adjustment unit 47 raises the heating temperature for the pipe forming the gas supply line L1 (S17).

At this time, it is preferable to return the values adjusted in S11, S13, and S15 to the original setting values prior to the adjustment, before raising the heating temperature for the pipe in S17. It is, however, also possible to raise the heating temperature for the pipe in S17 without returning some or all of the values adjusted in S11, S13, and S15 to the settings prior to the adjustment.

The adjustment unit 47 then determines whether there is any improvement in the side reaction, in the same manner as in S12 (S18), and ends the adjustment if there is some improvement in the side reaction.

By contrast, if it is determined that there is no improvement in the side reaction in S18, the adjustment unit 47 raises the heating temperature for the vaporizer 10 (S19).

The adjustment unit 47 then determines whether there is any improvement in the side reaction as in S12 (S20), and ends the adjustment if there is some improvement in the side reaction.

By contrast, if it is determined that there is no improvement in the side reaction in S20, the adjustment unit 47 then raises the concentration of the solution (S21), and ends the adjustment process.

Note that the adjustment unit 47 does not need to make all of the adjustments in S11, S13, S15, S17, S19, and S21, and some or all of these adjustments may be manually made by a user.

With the gas analysis device 100 according to the present embodiment having the configuration described above, the first actual concentration and the first ideal concentration are compared, the second actual concentration and the second ideal concentration are compared, and the parameter to be changed is determined and output, based on these comparison results. Therefore, by adjusting the setting value of the parameter to be changed, it is possible to suppress the progress of the side reaction that is presumed to be taking place.

As a result, the vaporization efficiency can be improved, and the actual concentration of the process gas can be brought closer to the ideal concentration.

In addition, because the information processing unit 40 has a function as the adjustment unit 47, it is possible to automate the adjustment of the setting value of the parameter to be changed.

Note that the present invention is not limited to the first embodiment described above.

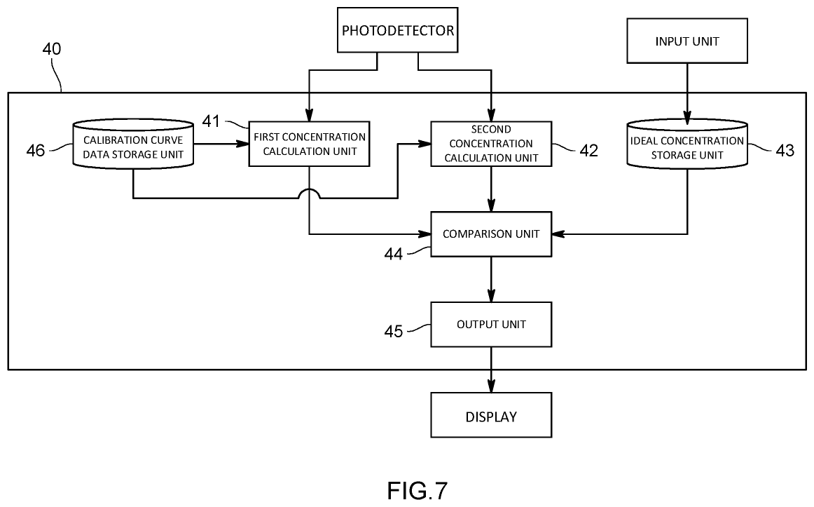

For example, the output unit 45 outputs the parameter to be changed to the adjustment unit 47 in the first embodiment, but may also display and output the parameter to be changed, on a display or the like, as illustrated in FIG. 7.

With such a configuration, it is possible to have the user recognize the parameter to be changed, in order to improve the vaporization efficiency. The user can then improve the vaporization efficiency by changing the setting value of the parameter to be changed, based on his/her experience, for example.

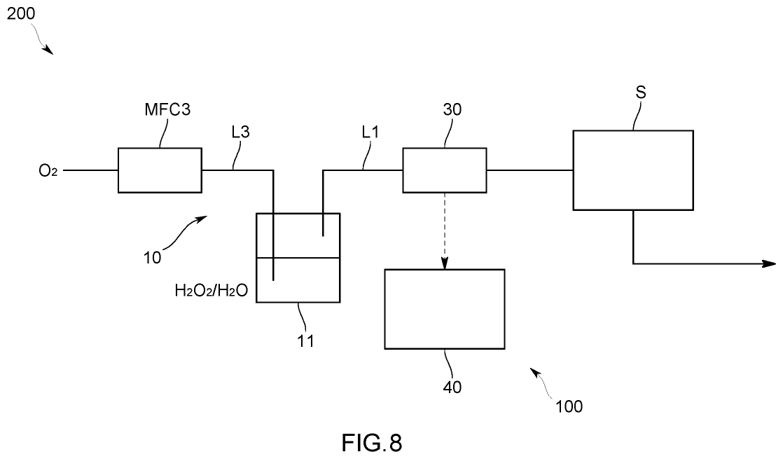

In the first embodiment, the fluid control system 200 uses the nozzle to eject and to vaporize the liquid material. However, as illustrated in FIG. 8, the fluid control system 200 may be a bubbler that vaporizes the liquid material or the molten solid material, by heating and bubbling the liquid material or the melted solid material.

Specifically, the fluid control system 200 includes a vaporization tank 11 that stores therein a liquid material or a solid material, a carrier gas feed line L3 for feeding a carrier gas that is for bubbling the material and vaporizing the material to the vaporization tank 11, a mass flow controller MFC3 that is a fluid controller provided in the carrier gas feed line L3, and a gas supply line L1 that supplies the gas vaporized in the vaporization tank 11 to the gas supply space S such as a chamber. In other words, in this embodiment, the vaporization tank 11 and the carrier gas feed line L3 function as the vaporizer 10 that vaporizes the liquid material or the solid material.

In this configuration, examples of the devices making up the fluid control system 200 include the vaporizer 10, the mass flow controller MFC 3, a pipe serving as the gas supply line L1, a pipe forming the carrier gas feed line L3, and a heater for heating these pipes.

Examples of the parameter to be changed include a temperature of the solution in the vaporization tank 11, a solution concentration in the vaporization tank 11, a heating temperature for the vaporizer 10, a flow rate setting of the vaporizer 10, a heating temperature for the pipe serving as the gas supply line L1, and a heating temperature for the pipe forming the carrier gas feed line L3. In the configuration of FIG. 8, the flow rate setting of the vaporizer 10 is a flow rate setting of the mass flow controller MFC3.

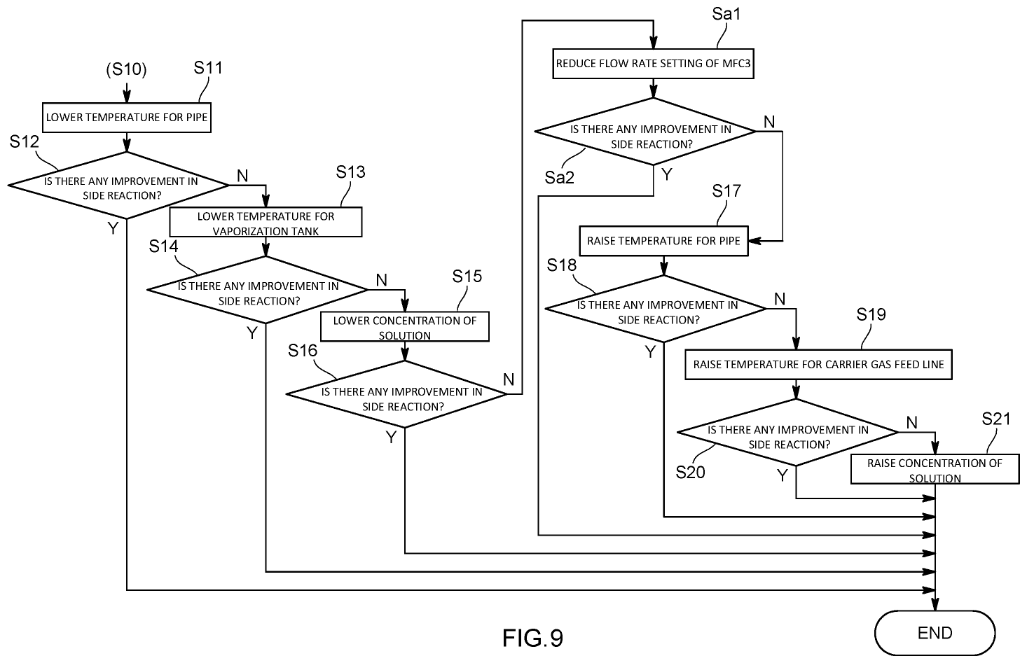

As an operation of the information processing unit 40 in the configuration illustrated in FIG. 8, S1 to S9 that are the same as those in FIG. 5 according to the embodiment described above may be used. Therefore, an example of the operation subsequent to the outputting the various parameters to be changed in S10 will be described with reference to FIG. 9, by using an example in which hydrogen peroxide water is used. Note that descriptions of the operations that are the same as those in FIG. 6, e.g., the step of determining whether there is any improvement in the side reaction, will be omitted.

To begin with, in order to suppress decomposition, the adjustment unit 47 lowers the heating temperature for the pipe of the gas supply line L1 (S11).

If it is then determined that there is no improvement in the side reaction, the adjustment unit 47 lowers the temperature of the solution in the vaporization tank 11 (S13). If it is determined that there is still not improvement in the side reaction, the adjustment unit 47 lowers the concentration of the solution in the vaporization tank 11 (S15).

If it is determined that there is still no improvement in the side reaction, the adjustment unit 47 reduces the flow rate setting of the mass flow controller MFC3 (Sa1) to check whether a reduction in the amount of hydrogen peroxide water sent to the gas supply line L1 contributes to suppression of the decomposition reaction.

The adjustment unit 47 then determines whether there is any improvement in the side reaction (Sa2). If there is no improvement in the side reaction, the adjustment unit 47 raises the heating temperature for the gas supply line L1, to suppress the liquefaction and/or the redissolution (S17).

If it is then determined that there is no improvement in the side reaction, the adjustment unit 47 raises the heating temperature for the carrier gas feed line L3, to suppress the oxygen gas from depriving heat from the hydrogen peroxide gas and the H₂O gas flowing through the gas supply line L1 (S19). If it is determined that there is still no improvement in the side reaction, the adjustment unit 47 raises the concentration of the solution in the vaporization tank 11 (S21).

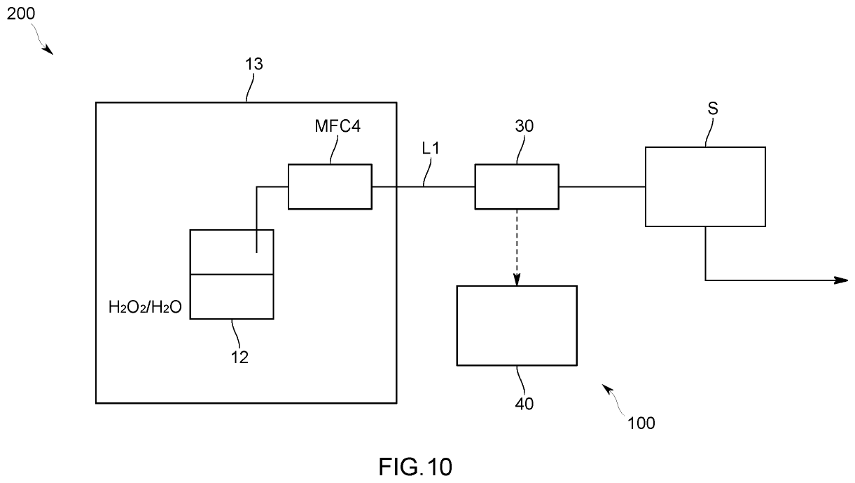

Furthermore, as illustrated in FIG. 10, the fluid control system 200 may be a baking vaporizer 12 storing therein a liquid material or a solid material is placed in a thermostatic bath 13, to vaporize the liquid material or the solid material. Note that the it is not necessarily required for the thermostatic bath 13 to be provided.

Specifically, the fluid control system 200 includes the vaporizer 12 and the thermostatic bath 13 described above, the gas supply line L1 that supplies the gas vaporized by the vaporizer 12 into the gas supply space S such as a chamber, and a mass flow controller MFC4 as a fluid controller provided in the gas supply line L1 and disposed in the thermostatic bath 13.

In this configuration, examples of devices making up the fluid control system include a pipe forming the vaporizer 12, the thermostatic bath 13, the mass flow controller MFC4, and the gas supply line L1, and the heater for heating the pipe.

Examples of the parameter to be changed include the heating temperature for the vaporizer 12, the flow rate setting of the vaporizer 12, the temperature setting of the thermostatic bath 13, and the heating temperature for the pipe serving as the gas supply line L1. In the configuration of FIG. 10, the flow rate setting of the vaporizer 12 is a flow rate setting of the mass flow controller MFC4.

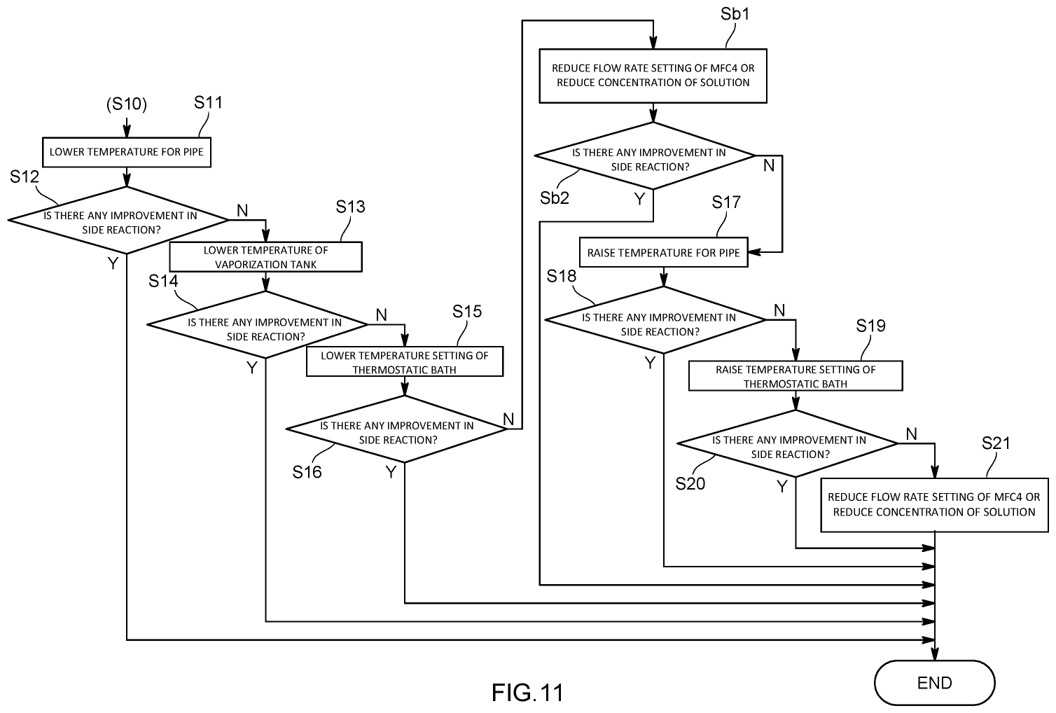

As an operation of the information processing unit 40 in the configuration illustrated in FIGS. 10, S1 to S9 that are the same as those in FIG. 5 according to the embodiment described above may be used. Therefore, an example of the operation subsequent to the outputting the various parameters to be changed in S10 will be described with reference to FIG. 11, by using an example in which hydrogen peroxide water is used. Note that descriptions of the operations that are the same as those in FIG. 6, e.g., the step of determining whether there is any improvement in the side reaction, will be omitted.

To begin with, in order to suppress decomposition, the adjustment unit 47 lowers the heating temperature for the pipe of the gas supply line L1 (S11).

If it is then determined that there is no improvement in the side reaction, the adjustment unit 47 lowers the temperature of the solution in the vaporizer 12 (S13). If it is determined that there is still no improvement in the side reaction, the adjustment unit 47 lowers the temperature setting of the thermostatic bath 13 (S15).

If it is determined that there is still no improvement in the side reaction, the adjustment unit 47 either reduces the flow rate setting of the mass flow controller MFC4 or reduces the concentration of the solution in the vaporizer 12 (Sb1) to check whether a reduction in the amount of the hydrogen peroxide water sent to the gas supply line L1 contributes to suppression of the decomposition reaction.

The adjustment unit 47 then determines whether there is any improvement in the side reaction (Sb2). If there is no improvement in the side reaction, the adjustment unit 47 raises the heating temperature for the gas supply line L1, to suppress the liquefaction and/or the redissolution (S17).

If it is then determined that there is no improvement in the side reaction, the adjustment unit 47 increases the temperature setting of the thermostatic bath 13 (S19). If it is determined that there is still no improvement in the side reaction, the adjustment unit 47 reduces the flow rate setting of the mass flow controller MFC4 or lowers the concentration of the solution in the vaporizer 12 (S21), considering the possibility that the hydrogen peroxide gas and/or the H₂O gas exerting a pressure equal to or higher than an equilibrium vapor pressure is flowing through the gas supply line L1.

In the first embodiment, the liquid material is a mixture of hydrogen peroxide (H₂O₂) and water (H₂O). However, the liquid material may be a mixture of formaldehyde and water, a mixture of peracetic acid and water. Furthermore, various liquid materials may be used without limitation to an aqueous solution.

Examples of the solid material include W(CO)₆ (tungsten hexacarbonyl).

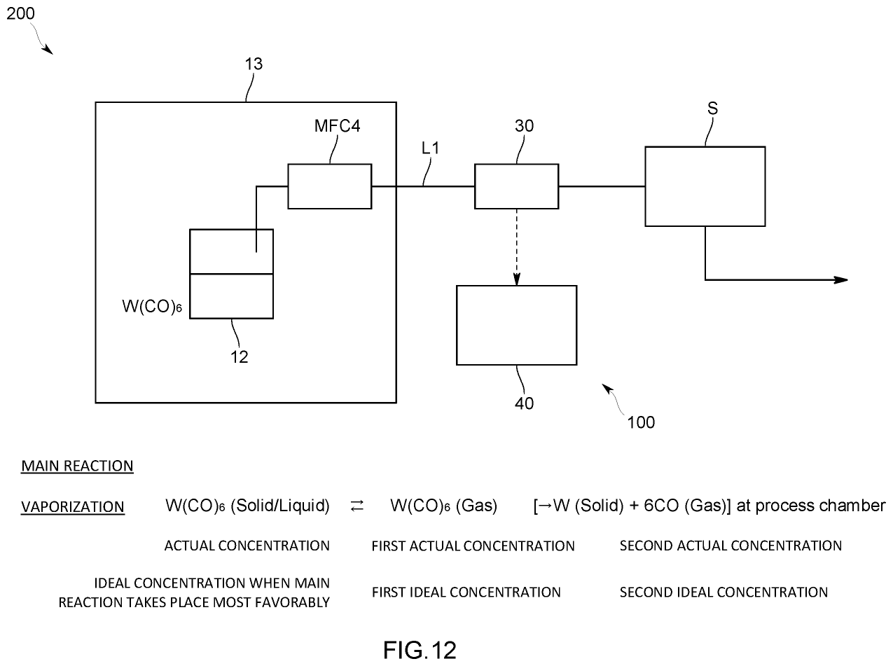

When this solid material is used, the main reaction is a reaction taking place in the vaporization tank 12 and the gas supply space S that is a process chamber. Specifically, as illustrated in FIG. 12, W(CO)₆ gas is generated as a process gas in the vaporization tank 12, and CO gas is generated as a by-product gas in the process chamber.

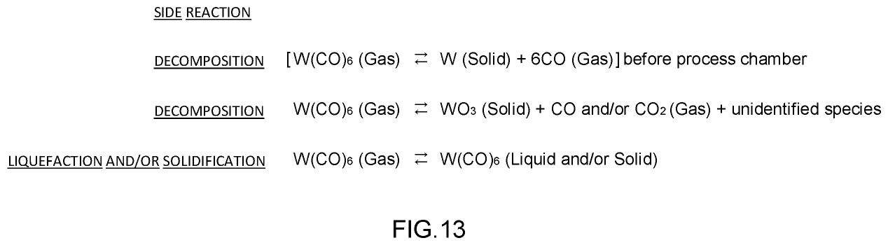

Examples of the side reactions that can take place when this solid material is used include, as illustrated in FIG. 13, decomposition of the process gas taking place before the process gas reaches the process chamber, decomposition of the process gas taking place separately from such a decomposition, and liquefaction and/or solidification of the process gas.

When the main reaction is taking place most favorably, the process gas does not become decomposed before reaching the process chamber, and therefore, no CO gas that is a by-product gas will be flowing through the gas supply line L1. In other words, the second ideal concentration should be zero. In other words, in this embodiment, the by-product gas is not generated in the main reaction if the main reaction is taking place most favorably, but is generated only in a side reaction.

Thus, as illustrated in FIG. 12, the concentration monitor 30 in this example is responsible for checking that no CO gas is flowing through the gas supply line L1.

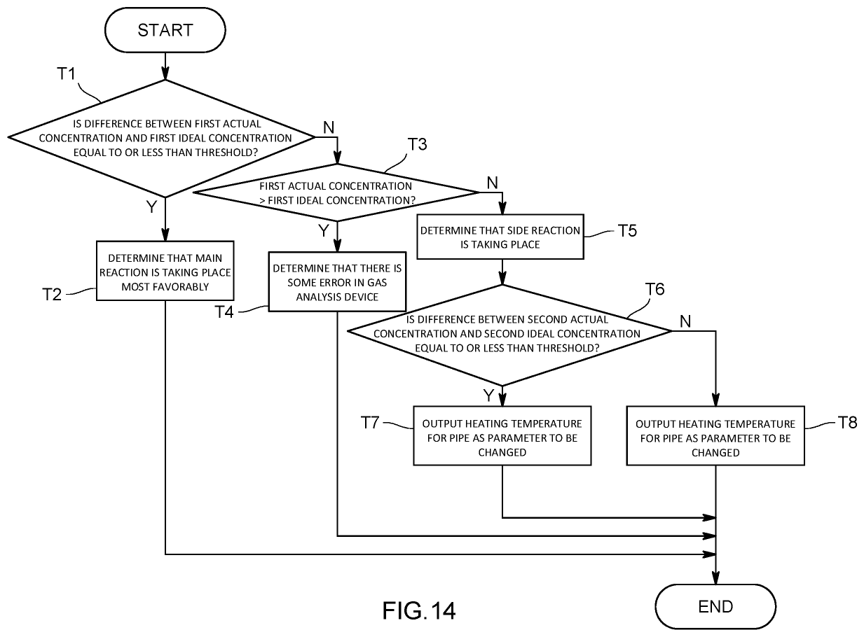

The flowchart illustrated in FIG. 14 is one example of the operation of the information processing unit in this embodiment. Note that the sequence of from T1 to T6 is the same as the sequence from S1 to S6 in the first embodiment, and therefore, detailed descriptions thereof will be omitted.

If the comparison result of the comparison unit 44 in T6 indicates that the difference between the second actual concentration and the second ideal concentration is equal to or less than the threshold, it is highly likely that liquefaction and/or solidification of the process gas flowing through the gas supply line L1 is taking place as a side reaction, because the second ideal concentration is zero, as mentioned earlier.

Therefore, when this is applicable, that is, if the comparison result of the comparison unit 44 in T3 indicates that the first actual concentration is lower than the first ideal concentration, and the comparison result of the comparison unit 44 in T6 indicates that the difference between the second actual concentration and the second ideal concentration is equal to or less than the threshold, the output unit 45 outputs the heating temperature for the pipe forming the gas supply line L1 as the parameter to be changed (T7). By increasing the heating temperature for the pipe, it is possible to suppress liquefaction and/or solidification that is presumed to be taking place as a side reaction.

By contrast, if the comparison result in T6 indicates that the difference between the second actual concentration and the second ideal concentration is greater than the threshold, it is highly likely that decomposition of the process gas is taking place as a side reaction.

Therefore, when this is applicable, that is, if the comparison result of the comparison unit 44 in T3 indicates that the first actual concentration is lower than the first ideal concentration, and the comparison result of the comparison unit 44 in T6 indicates that the difference between the second actual concentration and the second ideal concentration is greater than the threshold, the output unit 45 outputs the heating temperature for the pipe of the gas supply line L1 as the parameter to be changed (T8). By lowering the heating temperature for the pipe, it is possible to suppress decomposition of the process gas that is presumed to be taking place as a side reaction.

In addition, some of the functions of the first embodiment may be executed by a machine learning unit that performs an operation using a machine learning algorithm. For example, a machine learning unit may be caused to execute one or both of the functions of the output unit 45 and the adjustment unit 47.

### Second Embodiment

A second embodiment of the gas analysis device according to the present invention will now be explained.

A gas analysis device according to the present embodiment is a gas analysis device that analyzes a compound gas generated in a main reaction that is vaporization of a solid material, and a by-product gas generated in a side reaction that is different from the main reaction, and the configuration and the operation of the information processing unit are different from those of the embodiment described above. Therefore, these differences will be described below in detail.

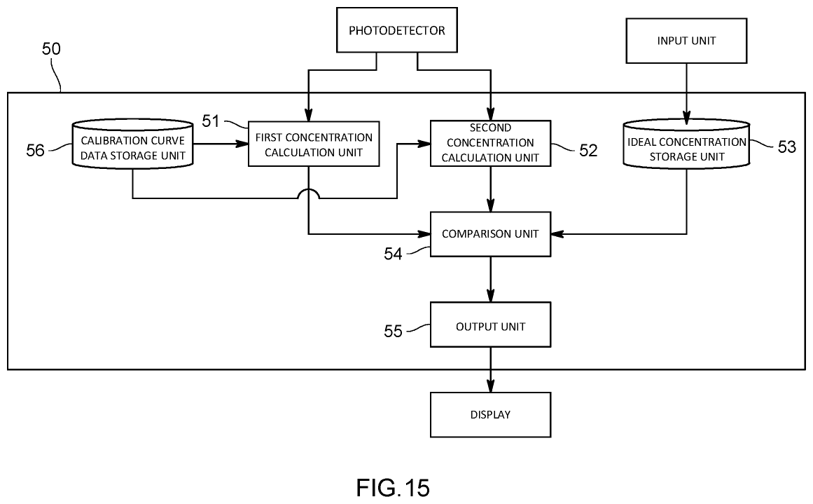

As illustrated in FIG. 15, the information processing unit 50 according to the present embodiment functions as a first concentration calculation unit 51, a second concentration calculation unit 52, an ideal concentration storage unit 53, a comparison unit 54, and an output unit 55. The concentration of the gas described below may be a concentration of the component in the gas or a partial pressure of the gas.

An operation of this information processing unit 50 according to the present embodiment will now be explained, also as an explanation of the function of each unit.

The first concentration calculation unit 51 calculates the concentration (hereinafter, also referred to as a first actual concentration) of the W(CO)₆ gas that is a compound gas. Specifically, the first concentration calculation unit 51 receives a light intensity signal that is an output signal from the photodetector, and performs an operation on the value indicated by the light intensity signal, to calculate the concentration of the W(CO)₆ gas contained in the gas flowing through the gas supply line L1, as the first actual concentration. Note that this operation uses first calibration curve data indicating the relationship between the value specified in the light intensity signal and the first actual concentration, and the first calibration curve data is stored in a calibration curve data storage unit 56 set in a predetermined area of the memory (see FIG. 15).

The second concentration calculation unit 52 calculates the concentration (hereinafter, also referred to as a second actual concentration) of CO gas that is a by-product gas. Specifically, the second concentration calculation unit 52 receives a light intensity signal that is an output signal from the photodetector, performs operation on a value indicated by the light intensity signal, and calculates the concentration of CO gas contained in the gas flowing through the gas supply line L1 as the second actual concentration. Note that this operation uses second calibration curve data indicating the relationship between the value specified in the light intensity signal and the second actual concentration, and the second calibration curve data is stored in the calibration curve data storage unit 56 set in a predetermined area of the memory (see FIG. 15).

In the present embodiment, the first concentration calculation unit 51 and the second concentration calculation unit 52 are configured to calculate the first actual concentration and the second actual concentration, respectively, based on output signals from the common photodetector. In this manner, reductions in the size as well as the manufacturing cost of the device can be achieved. However, it is also possible to configure the first concentration calculation unit 51 and the second concentration calculation unit 52 to calculate the first actual concentration and the second actual concentration, respectively, based on output signals from the different photodetectors, respectively.

The ideal concentration storage unit 53 is set in a predetermined area of the memory, and stores therein a first ideal concentration that is the ideal concentration of the W(CO)₆ gas achieved when the main reaction described above takes place most favorably, and a second ideal concentration that is the concentration of the CO gas also achieved when the main reaction described above takes place most favorably.

The first ideal concentration may be obtained in advance, through a calculation, e.g., before control process of the fluid control system 200 starts. Specifically, in the configuration in which the W(CO)₆ gas as the process gas is pressure-fed by the carrier gas, as illustrated in FIGS. 1 and 8, the first ideal concentration may be calculated from a volume fraction, as a theoretical concentration, using the total flow rate of the gas flowing through the concentration monitor 30, and the flow rate setting of the carrier gas. By contrast, as illustrated in FIG. 12, in the configuration not using the carrier gas, the theoretical concentration of the W(CO)₆ gas is 100%.

In the manner described above, the theoretical concentration may be set to the first ideal concentration. However, because the vaporization in the present embodiment is a process in which a solid material is heated in a high vacuum, a high load is applied to a substance, and decomposition may take place simultaneously with the main reaction described above. If so, the concentration (effective concentration) measured by the concentration monitor 30 deviates from the theoretical concentration, so it is possible to obtain a ratio of the effective concentration to the theoretical concentration in advance, and to set the concentration obtained by multiplying the ratio to the theoretical concentration as the first ideal concentration. Alternatively, it is also possible not to obtain the ratio, and to set the effective concentration to the first ideal concentration.

In the same manner as the first ideal concentration, the second ideal concentration may be obtained in advance, through a calculation, e.g., before the control process of the fluid control system 200 starts. In this embodiment, when the main reaction takes place most favorably, no CO gas is generated, and therefore, the theoretical concentration, which is theoretically calculated, is 0%.

This theoretical concentration may be set to the second ideal concentration, or the second ideal concentration may be set to about a few percent in the volume fraction, considering the decomposition taking place under a high vacuum, as mentioned above.

The first ideal concentration and the second ideal concentration thus calculated are input from the external via an input unit, for example, and stored in the ideal concentration storage unit 53. It is also possible to provide the information processing unit 50 with the function as the ideal concentration calculation unit that calculates the first ideal concentration and the second ideal concentration, and to store the first ideal concentration and the second ideal concentration calculated by the ideal concentration calculation unit in the ideal concentration storage unit 53.

The comparison unit 54 compares the first actual concentration and the first ideal concentration, and compares the second actual concentration and the second ideal concentration. Specifically, the comparison unit 54 determines a quantitative relationship between the first actual concentration and the first ideal concentration, and determines a quantitative relationship between the second actual concentration and the second ideal concentration.

The comparison unit 54 according to the present embodiment is configured to compare the first actual concentration and the first ideal concentration to determine whether a side reaction different from the main reaction is taking place, and to determine whether there is any error in the device.

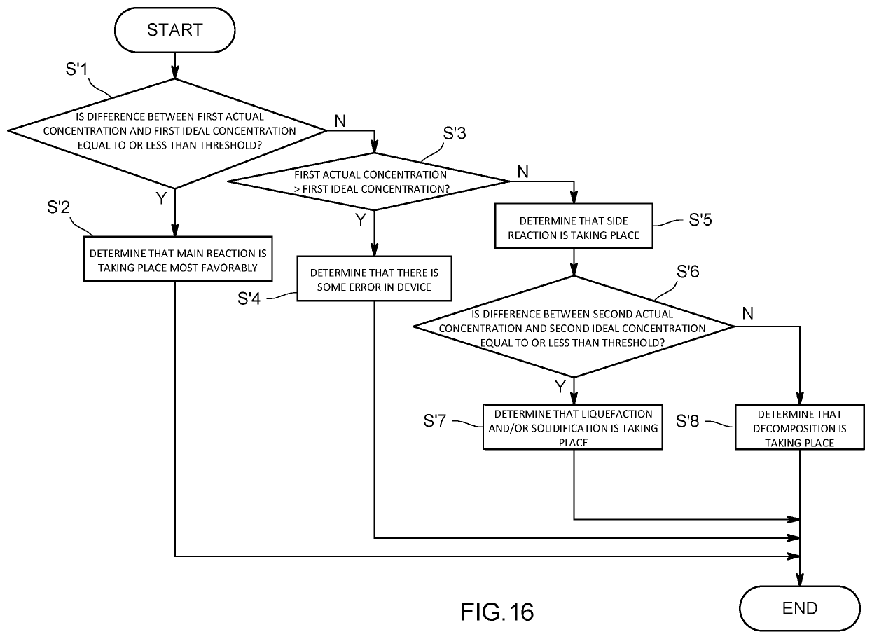

More specifically, as illustrated in FIG. 16, the comparison unit 54 first compares the first actual concentration and the first ideal concentration (S′1). When the difference between the first actual concentration and the first ideal concentration is equal to or less than the predetermined threshold, the comparison unit 54 determines that the main reaction is taking place most favorably (S′2).

By contrast, if the difference between the first actual concentration and the first ideal concentration is greater than the predetermined threshold in S′1, the comparison unit 54 determines the quantitative relationship between the first actual concentration and the first ideal concentration (S′3), and determines whether a side reaction that is different from the main reaction is taking place, or whether there is any error in the device (S′4, S′5).

Specifically, if the first actual concentration is higher than the first ideal concentration, the comparison unit 54 determines that there is some error in the device (S′4). Examples of the errors include a calibration error and setting errors of various setting values, such as the first calibration curve data, the second calibration curve data, and the vaporization efficiency described above.

By contrast, if the first actual concentration is lower than the first ideal concentration, the comparison unit 54 determines that a side reaction that is different from the main reaction is taking place (S′5).

If it is determined that a side reaction is taking place in S′5, the comparison unit 54 identifies the type of side reaction, based on the comparison result between the second actual concentration and the second ideal concentration. As described above, examples of the type of side reaction include decomposition of the W(CO)₆ gas taking place before the gas reaches the process chamber, decomposition of the W(CO)₆ gas taking place separately from such a decomposition, and liquefaction and/or solidification of the W(CO)₆ gas (see FIG. 13). The type of side reaction identified by the comparison unit 54 may include at least one of the decomposition, liquefaction, and solidification.

The comparison unit 54 according to the present embodiment compares the second actual concentration and the second ideal concentration (S′6). If the difference between the second actual concentration and the second ideal concentration is equal to or less than a predetermined threshold, the comparison unit 54 determines that liquefaction and/or solidification of the W(CO)₆ gas is taking place as a side reaction (S′7).

By contrast, if the difference between the second actual concentration and the second ideal concentration is greater than the predetermined threshold in S′6, the comparison unit 54 determines that decomposition of the W(CO)₆ gas is taking place as a side reaction (S′8).

As described above, the analysis result obtained by the comparison unit 54 includes at least the result of the comparison between the first actual concentration and the first ideal concentration and the result of the comparison between the second actual concentration and the second ideal concentration. Further, the analysis results according to this embodiment include various determination results determined based on the comparison results, i.e., whether there is any error in the device, whether a side reaction that is different from the main reaction is taking place, and the type of side reaction that is taking place (decomposition, liquefaction, and/or solidification).

In the present embodiment, a storage unit such as an internal memory or an external memory of the information processing unit 50 stores therein determination information such as a table including candidates of the results of comparisons in S′1, S′3, and S′6 by the comparison unit 54 and one or more determination results associated with the candidates. The comparison unit 54 then derives one or more determination results, in each step described above, by using the determination information and the comparison result. Note that the storage unit described above may be a component of the information processing unit 50, may be a component of a computer that is different from the information processing unit 50 included in the fluid control system 200, or may be provided outside the fluid control system 200, such as a client server.

The analysis result based on the comparisons performed by the comparison unit 54 is then output visibly by the output unit 55. Specifically, the output unit 55 outputs some or all of the information included in the analysis result visibly, and is configured to display and output that there is some error in the device that a side reaction is taking place, and the type of the side reaction, on a display. Note that the output unit 55 may also print to output the analysis result on a paper sheet or the like.

With the gas analysis device 100 according to the present embodiment having the configuration described above, because the first actual concentration and the first ideal concentration, which are the concentrations of the W(CO)₆ gas, are compared and the analysis result is output, it is possible to recognize whether there is a difference between the first actual concentration and the first ideal concentration, that is, whether the main reaction is taking place most favorably.

In addition, because the second actual concentration and the second ideal concentration, which are the concentrations of CO gas, are also compared and the analysis result is output, it is easy to identify a highly probable cause, among various possible causes, such as errors in the device and a side reaction such as decomposition, liquefaction, or solidification of W(CO)₆ gas, which cannot be recognized merely by comparing the first actual concentration and the first ideal concentration, as a cause of the difference between the first actual concentration and the first ideal concentration, and it becomes easy to take an appropriate measure for reducing the difference between the first actual concentration and the first ideal concentration.

Note that the present invention is not limited to the embodiments described above.

For example, in the above embodiment, the output unit 55 outputs that there is some error in the device, that a side reaction is taking place, and the type of the side reaction, but may output only some of these. In addition, the output unit 55 may also display the result of a comparison (quantitative relationship) between the first actual concentration and the first ideal concentration and the result of a comparison (quantitative relationship) between the second actual concentration and the second ideal concentration. In this case, it is also possible for the comparison unit 54 not to determine whether there is any error in the device, whether a side reaction is taking place, and what type of the side reaction is taking place.

In addition, the output unit 55 may output the first actual concentration and the first ideal concentration in a comparable manner, without outputting the analysis result by the comparison unit 54, and output the second actual concentration and the second ideal concentration in a comparable manner on, for example, a display or the like. In this case, the information processing unit 50 may not have the function as the comparison unit 54.

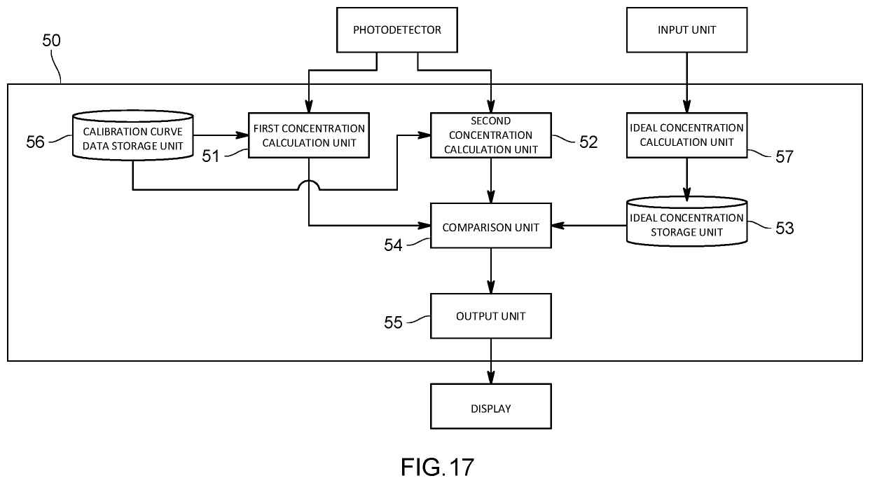

Furthermore, as described in the second embodiment, the information processing unit 50 may have a function as the ideal concentration calculation unit 57 that calculates the first ideal concentration and the second ideal concentration, as illustrated in FIG. 17. Specifically, as explained in the description of the first ideal concentration and the second ideal concentration in the embodiment, effective concentrations deviate from their theoretical concentrations as the decomposition takes place as a result of heating in a high vacuum. Therefore, by inputting a ratio between the theoretical concentration and the effective concentration in advance, for example, the ideal concentration calculation unit 57 can calculate the first and the second ideal concentrations using this ratio.

The information processing unit 50 may further include a function as a notification unit that, when a difference between the first actual concentration and the first ideal concentration is greater than a predetermined threshold, as a result of causing the comparison unit to compare the first actual concentration and the first ideal concentration, makes a notification of the difference.

Furthermore, some of the functions of the first concentration calculation unit 51, the second concentration calculation unit 52, the comparison unit 54, and the output unit 55 included in the information processing unit 50 may be provided in another computer, or the ideal concentration storage unit 53 may be set in a predetermined area of an external memory that is different from the memory of the information processing unit 50.

Furthermore, some of the functions of the second embodiment may be executed by a machine learning unit that performs an operation using a machine learning algorithm. For example, a machine learning unit may be caused to execute one or both of the functions of the comparison unit 54 and the output unit 55.

In addition, various modifications of the embodiments and combinations thereof may be made within the scope not deviating from the gist of the present invention.

### REFERENCE SIGNS LIST

100 Gas analysis device
200 Fluid control system
S Gas supply space
10 Vaporizer
L1 Gas supply line
30 Concentration monitor
40, 50 Information processing unit
41, 51 First concentration calculation unit
42, 52 Second concentration calculation unit
43, 53 Ideal concentration storage unit
44, 54 Comparison unit
45, 55 Output unit
46, 56 Calibration curve data storage unit
47 Adjustment unit

1. A gas analysis device that is used in a fluid control system that controls a process gas obtained by vaporizing a liquid material or a solid material, the gas analysis device comprising:
a first concentration calculation unit that calculates a concentration of the process gas;
a second concentration calculation unit that calculates a concentration of a by-product gas at least generated in a side reaction that is a reaction different from a main reaction for generating the process gas;
a comparison unit that compares a first actual concentration that is the concentration of the process gas calculated by the first concentration calculation unit with a first ideal concentration that is the concentration of the process gas achieved when the main reaction takes place most favorably, and compares a second actual concentration that is the concentration of the by-product gas calculated by the second concentration calculation unit with a second ideal concentration that is the concentration of the by-product gas achieved when the main reaction takes place most favorably; and
an output unit that determines a parameter to be changed, that is a parameter a setting value of which is to be changed, among parameters set in devices included in the fluid control system, based on comparison results of the comparison unit, and outputs the parameter to be changed.

2. The gas analysis device according to claim 1, wherein, the parameter to be changed is a heating temperature of a pipe through which the process gas flows, a heating temperature of a vaporizer that vaporizes the liquid material or the solid material, a flow rate setting of the vaporizer, or a concentration of the liquid material.

3. The gas analysis device according to claim 2, wherein, when a comparison result of the comparison unit indicates that the first actual concentration is lower than the first ideal concentration and a difference between the second actual concentration and the second ideal concentration is equal to or less than a threshold, the output unit outputs the heating temperature for the pipe as the parameter to be changed.

4. The gas analysis device according to claim 2, wherein, when a comparison result of the comparison unit indicates that the first actual concentration is lower than the first ideal concentration, a difference between the second actual concentration and the second ideal concentration is greater than a threshold, and the second actual concentration is higher than the second ideal concentration, the output unit outputs the heating temperature for the vaporizer or the flow rate setting of the vaporizer as the parameter to be changed.

5. The gas analysis device according to claim 2, wherein, when a comparison result of the comparison unit indicates that the first actual concentration is lower than the first ideal concentration, a difference between the second actual concentration and the second ideal concentration is greater than a threshold, and the second actual concentration is lower than the second ideal concentration, the output unit outputs the heating temperature for the pipe, the heating temperature for the vaporizer, the flow rate setting of the vaporizer, or the concentration of the liquid material, as the parameter to be changed.

6. The gas analysis device according to claim 1, further comprising an adjustment unit that receives the parameter to be changed output from the output unit, and adjusts a setting value of the parameter to be changed.

7. The gas analysis device according to claim 1, wherein the output unit visibly outputs the parameter to be changed.

8. The gas analysis device according to claim 1, wherein the first concentration calculation unit and the second concentration calculation unit calculate concentrations based on an output signal output from a common photodetector.

9. A fluid control system comprising:
a vaporizer that vaporizes the liquid material or solid material;
a fluid controller that controls the process gas, the liquid material, or carrier gas; and
the gas analysis device according to claim 1.

10. A computer-readable medium including a gas analysis program used in a fluid control system that controls a process gas obtained by vaporizing a liquid material or a solid material, the gas analysis program causing a computer to exert functions as:
a first concentration calculation unit that calculates a concentration of the process gas;
a second concentration calculation unit that calculates a concentration of a by-product gas at least generated in a side reaction that is a reaction different from a main reaction for generating the process gas;
a comparison unit that compares a first actual concentration that is the concentration of the process gas calculated by the first concentration calculation unit with a first ideal concentration that is the concentration of the process gas achieved when the main reaction takes place most favorably, and compares a second actual concentration that is the concentration of the by-product gas calculated by the second concentration calculation unit with a second ideal concentration that is the concentration of the by-product gas achieved when the main reaction takes place most favorably; and
an output unit that determines a parameter to be changed, that is a parameter a setting value of which is to be changed, among parameters set in devices included in the fluid control system, based on comparison results of the comparison unit, and outputs the parameter to be changed.

11. A gas analysis method that is used in a fluid control system that controls a process gas obtained by vaporizing a liquid material or a solid material, the gas analysis method comprising:
first calculating a concentration of the process gas;
second calculating a concentration of a by-product gas at least generated in a side reaction that is a reaction different from a main reaction for generating the process gas;
comparing a first actual concentration that is the concentration of the process gas calculated by the first concentration calculating with a first ideal concentration that is the concentration of the process gas achieved when the main reaction takes place most favorably, and comparing a second actual concentration that is the concentration of the by-product gas calculated by the second concentration calculating with a second ideal concentration that is the concentration of the by-product gas achieved when the main reaction takes place most favorably; and
outputting by determining a parameter to be changed, that is a parameter a setting value of which is to be changed, among parameters set in devices included in the fluid control system, based on comparison results of the comparing, and of outputting the parameter to be changed.

12. A gas analysis device that analyzes a compound gas generated in a main reaction in which a solid material is vaporized, and a by-product gas generated in a side reaction that is different from the main reaction, the gas analysis device comprising:
a first concentration calculation unit that calculates a concentration of the compound gas;
a second concentration calculation unit that calculates a concentration of the by-product gas;
a comparison unit that compares a first actual concentration that is the concentration of the compound gas calculated by the first concentration calculation unit, with a first ideal concentration that is the concentration of the compound gas achieved when the main reaction takes place most favorably, and compares a second actual concentration that is the concentration of the by-product gas calculated by the second concentration calculation unit with a second ideal concentration that is the concentration of the by-product gas achieved when the main reaction takes place most favorably; and
an output unit that outputs an analysis result based on comparisons performed by the comparison unit.

13. A computer-readable medium including a gas analysis program used in a gas analysis device that analyzes a compound gas generated in a main reaction in which a solid material is vaporized and a by-product gas generated in a side reaction different from the main reaction, the gas analysis program causing a computer to execute functions as:
a first concentration calculation unit that calculates a concentration of the compound gas;
a second concentration calculation unit that calculates a concentration of the by-product gas;
a comparison unit that compares a first actual concentration that is the concentration of the compound gas calculated by the first concentration calculation unit, with a first ideal concentration that is the concentration of the compound gas achieved when the main reaction takes place most favorably, and compares a second actual concentration that is the concentration of the by-product gas calculated by the second concentration calculation unit with a second ideal concentration that is the concentration of the by-product gas achieved when the main reaction takes place most favorably; and
an output unit that outputs an analysis result based on comparisons performed by the comparison unit.

14. A gas analysis method for analyzing a compound gas generated in a main reaction in which a solid material is vaporized and a by-product gas generated in a side reaction that is different from the main reaction, the gas analysis method comprising:
analyzing by comparing a first actual concentration that is a calculated concentration of the compound gas, with a first ideal concentration that is the concentration of the compound gas achieved when the main reaction takes place most favorably, and comparing a second actual concentration that is a calculated concentration of the by-product gas, with a second ideal concentration that is the concentration of the by-product gas achieved when the main reaction takes place most favorably; and
outputting an analysis result based on comparisons in the analyzing.

15. A gas analysis device that analyzes a compound gas and a by-product gas generated in a main reaction in which a solid material is vaporized, the gas analysis device comprising:
a first concentration calculation unit that calculates a concentration of the compound gas;
a second concentration calculation unit that calculates a concentration of the by-product gas; and
an output unit that outputs a first actual concentration that is a concentration of the compound gas calculated by the first concentration calculation unit, and a first ideal concentration that is a concentration of the compound gas when the main reaction takes place most favorably, in a comparable manner, and outputs a second actual concentration that is a concentration of the by-product gas calculated by the second concentration calculation unit, and a second ideal concentration that is the concentration of the by-product gas achieved when the main reaction takes place most favorably, in a comparable manner.
